(19) United States
(12) Patent Application Publication
(10) Pub. No.: US 2025/0312371 A1
(43) Pub. Date: Oct. 9, 2025

(54) CROSS-LINKED POLYMERIC CHELATORS COMPOSITIONS AND USE THEREOF

(71) Applicant: THE UNIVERSITY OF KANSAS, Lawrence, KS (US)

(72) Inventors: Cory BERKLAND, St. Louis, MO (US); Jian QIAN, Belle Mead, NJ (US)

(21) Appl. No.: 18/683,240

(22) PCT Filed: Aug. 12, 2022
(86) PCT No.: PCT/US22/40250

§ 371 Date: Feb. 12, 2024

Related U.S. Application Data
(60) Provisional application No. 63/233,024, filed on Aug. 13, 2021.
(60) Provisional application No. 63/316,831, filed on Mar. 4, 2022.

(51) Int. Cl. A61K 31/785; A61K 45/06

(52) U.S. Cl. CPC A61K 31/785; A61K 45/06

(57) ABSTRACT

Compositions comprising (i) a cross-linked polymeric chelator comprising a plurality of cross-linked polyamine polymer backbone chains and one or more chelators covalently coupled thereto, and (ii) an agent selected from an antacid, a histamine H2-receptor antagonist, a proton pump inhibitor, and a combination thereof. Also disclosed are methods of using a composition comprising the cross-linked polymeric chelator in combination with the agent, e.g., for removing metal from a medium or treating iron overload disease.

### CROSS REFERENCE TO RELATED APPLICATIONS

The present application claims priority to U.S. Provisional Application No. 63/233,024, filed Aug. 13, 2021; and claims priority to U.S. Provisional Application No. 63/316,831, filed Mar. 4, 2022, the entirety of which are incorporated herein by reference.

### BACKGROUND

Metals such as cadmium, lead, and arsenic are highly toxic to living organisms. Wastewater discharge may be a primary source of heavy metal release into the environment. The removal of heavy metal ions from industrial wastewater has been given much attention in the last decade, because such components can accumulate in living organisms. Upon their accumulation in the human body, these toxic metals may cause kidney failure, nerve system damage, and bone damage, as well as other serious diseases. The necessity to reduce the amount of heavy metal ions from the environment has led to an increasing interest in technologies that selectively remove such toxic metals.

There are 35 metals that are of concern because of occupational or residential exposure; 23 of these are the heavy elements or “heavy metals.” Heavy metals are chemical elements with a specific gravity that is at least 5 times the specific gravity of water. Small amounts of these elements are common in our environment and diet and in some cases are actually necessary for good health, but large amounts of any of them may cause acute or chronic toxicity. Heavy metal toxicity can result in damaged or reduced mental and central nervous function, lower energy levels, and damage to blood composition, lungs, kidneys, liver, and other vital organs. Long-term exposure may result in slowly progressing physical, muscular, and neurological degenerative processes that mimic Alzheimer's disease, Parkinson's disease, muscular dystrophy, and multiple sclerosis. Allergies are not uncommon and repeated long-term contact with some metals or their compounds may even cause cancer.

A particular heavy metal of concern is iron. Iron is an essential and ubiquitous element in all forms of life involved in a multitude of biological processes and essential for many critical human biological processes. Yet, the presence of excess iron in the body may lead to toxic effects.

Iron overload is a serious complication in patients that have 0-thalassemia and is the focal point of its management. In patients that do not receive transfusions, abnormal iron absorption can produce an increase in the body iron burden, which is evaluated to be in the 2-5 gram per year range. Patients that receive treatments that include regular blood transfusions can lead to double this amount of iron accumulation. Iron accumulation introduces progressive damage in liver, heart, and in the endocrine system if left untreated. The available iron is deposited in parenchymal tissues and in reticuloendothelial cells. When the iron load increases, the iron binding capacity of serum transferrin is exceeded and a non-transferrin-bound fraction of plasma iron (NTBI) appears. The NTBI can generate free hydroxyl radicals and induces dangerous tissue damage. Iron accumulates at different rates in various organs, each of which react in a characteristic way to the damage induced by NTBI and by the intracellular labile iron pool (LIP).

Current treatments for iron overload diseases include chelation therapy to chelate the iron and reduce its bioavailability. In one example, chelation therapy can be performed with desferoxamine (DFO), which is administered by subcutaneous infusion. Drugs that can be administered orally include deferiprone and Exjade. DFO therapy has reportedly been associated with several drawbacks including a narrow therapeutic window and lack of oral bioavailability. As a result, it requires administration for 8-12 hours per day by infusion. DFO cannot be readily absorbed through the intestine and must be injected intravenously thus, is not an ideal chelator since systemic side effects have been reported. Furthermore, concerns have arisen over its use due to numerous significant drug-related toxicities. Serious adverse effects such as neutropenia, agranulocytosis, hypersensitivity reactions, and blood vessel inflammation have also been reported upon the oral application of deferiprone and Exjade.

One possible method of avoiding the use of systemic iron chelators is to inhibit iron absorption from the gastrointestinal tract by orally available iron chelators that selectively sequester and remove excess dietary iron from the GI tract. Non-absorbed polymer therapies that act by sequestering a number of undesired ionic species in the gastrointestinal tract have been successful clinically. Using non-absorbed polymer therapies is particularly relevant to thalassemia intermedia and hemochromatosis. Iron binding polymers have considerable potential in this therapeutic approach as they can effectively bind iron to form nontoxic, inert complexes that are not absorbed by the gastrointestinal tract, thereby reducing the absorption of iron from the intestine.

Microorganisms have developed a sophisticated Fe(III) acquisition and transport systems involving siderophores. Siderophores are low molecular weight chelating agents that bind Fe(III) ion with high specificity. The iron binding affinity of siderophores dramatically exceeds that of iron chelating therapeutics currently available. Enterobactin, a naturally occurring tris-catechol siderophore, is the most powerful Fe(III) chelator known with a relative iron binding constant of 35.5. Since nearly all iron is absorbed in the gastrointestinal tract, next generation iron chelators must achieve significantly higher iron binding and selectivity with low toxicity and side effects.

Researchers have synthesized small molecule siderophore mimetics; however, compounds that directly mimic siderophores would be expected to enhance bacterial recruitment of iron. What is needed is a novel iron chelator that binds iron tightly and removes it from the body.

### SUMMARY

Generally, the present invention includes new compositions and systems for chelation of metals. In some embodiments, the present disclosure provides a composition comprising a crosslinked polymeric chelator. The polymeric chelator can include a polymer coupled with a metal chelator, and an agent such as an antacid, a histamine H2-receptor agonist, a proton pump inhibitor, or a combination thereof.

In one aspect, provided is composition comprising (i) a polymeric chelator comprising a plurality of polyamine polymer backbone chains and one or more chelators; and (ii) an agent selected from an antacid, a histamine H2-receptor antagonist, a proton pump inhibitor, and a combination thereof, wherein the one or more chelators are covalently coupled to one or more primary and/or secondary amines (e.g., one or more primary amines) of at least one of the plurality of polyamine polymer backbone chains; and wherein the plurality of polyamine polymer backbone chains are cross-linked with a plurality of cross-linkers. Embodiments of the cross-linked polymeric chelators include the following, alone or in any combination.

The composition wherein each of the plurality of polyamine polymer backbone chains comprises a polyamine polymer having a molecular weight (weight average molecular weight; “M_{w}”) of 1-50 kDa (e.g., 2-30 kDa, 5-25 kDa, 10-20 kDa, 2, 1 kDa, 2 kDa, 3 kDa, 4 kDa, 5 kDa, 10 kDa, 15 kDa, 20 kDa, 25 kDa, 30 kDa, 35 kDa, 40 kDa, 45 kDa, or 50 kDa).

The composition wherein each of the polyamine polymer backbone chains comprises repeating monomeric units each having the structure:

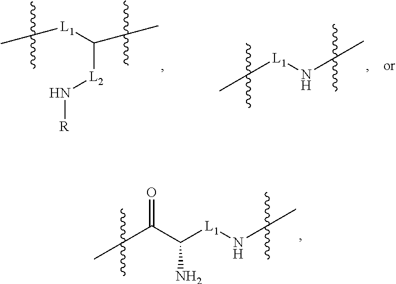

wherein L₁ is C₁-C₆alkylene, L₂ is a bond or C₁-C₆ alkylene, and R is H or C(O)R″ in which R″ is H, C₁-C₆ alkyl, or C₆-C₁₂ aryl. As used herein, “alkylene” refers to a divalent, straight-chained or branched, saturated hydrocarbon radical. As used herein, “alkyl” refers to a branched or straight-chain monovalent saturated aliphatic radical containing only C and H when unsubstituted. The term “aryl” as used herein, refers to any monocyclic or fused ring bicyclic system containing only carbon atoms in the ring(s), which has the characteristics of aromaticity in terms of electron distribution throughout the ring system.

The composition wherein the polyamine polymer backbone chains each comprise polyallylamine.

The composition wherein the polyamine polymer backbone chains each comprise poly(L-lysine).

The composition wherein the agent is an antacid.

The composition wherein the antacid is CaCO₃, NaHCO₃, MgCO₃, Mg(OH)₂, MgO, Al(OH)₃, or simethicone.

The composition wherein each of the plurality of cross-linkers is independently of structure:

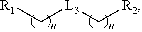

wherein R₁ and R₂ are independently selected from:

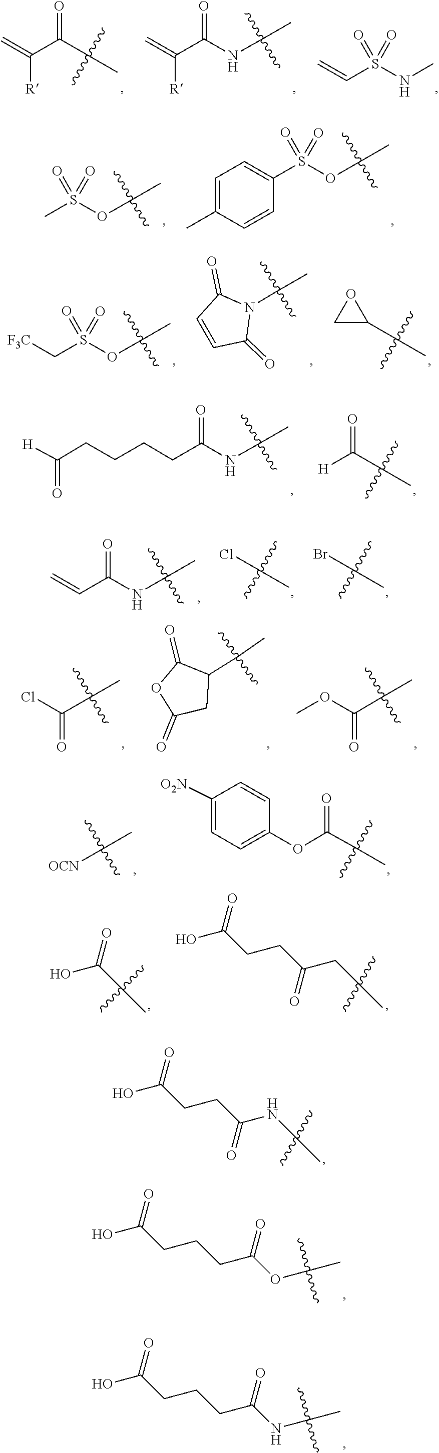

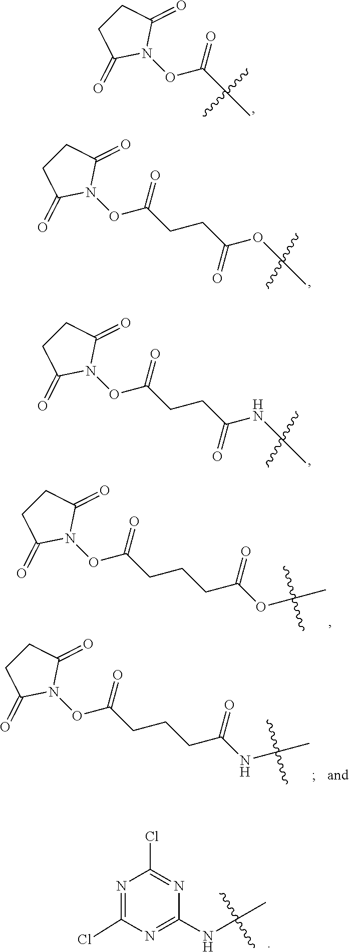 [SMILES: *C(=O)ON1C(=O)CCC1=O ; *OC(=O)CCC(=O)ON1C(=O)CCC1=O ; *NC(=O)CCC(=O)ON1C(=O)CCC1=O ; *OC(=O)CCCC(=O)ON1C(=O)CCC1=O ; *NC(=O)CCCC(=O)ON1C(=O)CCC1=O ; *Nc1nc(Cl)nc(CCl)n1]

R′ is C₁-C₆ alkyl; n is 0, 1, or 2; and L₃ is a bond, C₁-C₆alkylene, C₁-C₆heteroalkylene, C₃-C₈cycloalkylene, C₆-C₁₄arylene, or 5- or 6-membered heterocyclylene or polyethylene glycol. As used herein, “heteroalkylene” refers to a divalent, straight-chain or branched hydrocarbon radical in which one or more carbon atoms is replaced with a heteroatom (e.g., O, N, or S); “cycloalkylene” refers to a divalent, monocyclic hydrocarbon radical; “arylene” refers to a divalent, monocyclic, bicyclic, or multicyclic aromatic hydrocarbon radical; and “heterocyclylene” refers to a divalent, aromatic radical containing 1, 2, 3, or 4 heteroatoms as within the ring, and carbon atoms as the remaining ring atoms.

The composition wherein each of the plurality of cross-linkers is of structure:

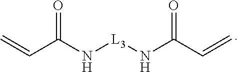

The composition wherein the polymeric chelator comprises a plurality of groups each having the structure:

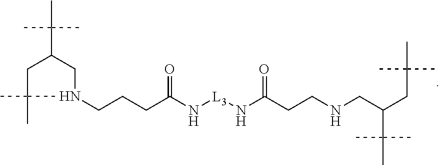

The composition wherein L₃ is methylene.

The composition wherein each of the plurality of cross-linkers is of structure:

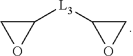

The composition wherein the polymeric chelator comprises a plurality of groups each having the structure:

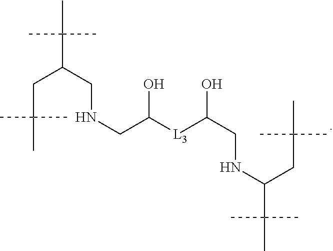

The composition wherein L₃ is polyethylene glycol.

The composition wherein the polyethylene glycol has a molecular weight (number average molecular weight; “Mₙ”) of 200 Daltons to 6000 Daltons (e.g., 400 Daltons to 6000 Daltons, 600 Daltons to 6000 Daltons, 1000 Daltons to 6000 Daltons, 2000 Daltons to 6000 Daltons, 400 Daltons to 2000 Daltons, 600 Daltons to 2000 Daltons, 1000 Daltons to 2000 Daltons, or 200 Daltons, 400 Daltons, 600 Daltons, 1000 Daltons, 2000 Daltons, or 6000 Daltons).

The composition wherein the polyethylene glycol has a molecular weight (Mₙ) of 400 Daltons to 2500 Daltons.

The composition wherein the polyethylene glycol has a molecular weight (Mₙ) of 800 Daltons and 2200 Daltons.

The composition wherein each of the plurality of cross-linkers is selected from:

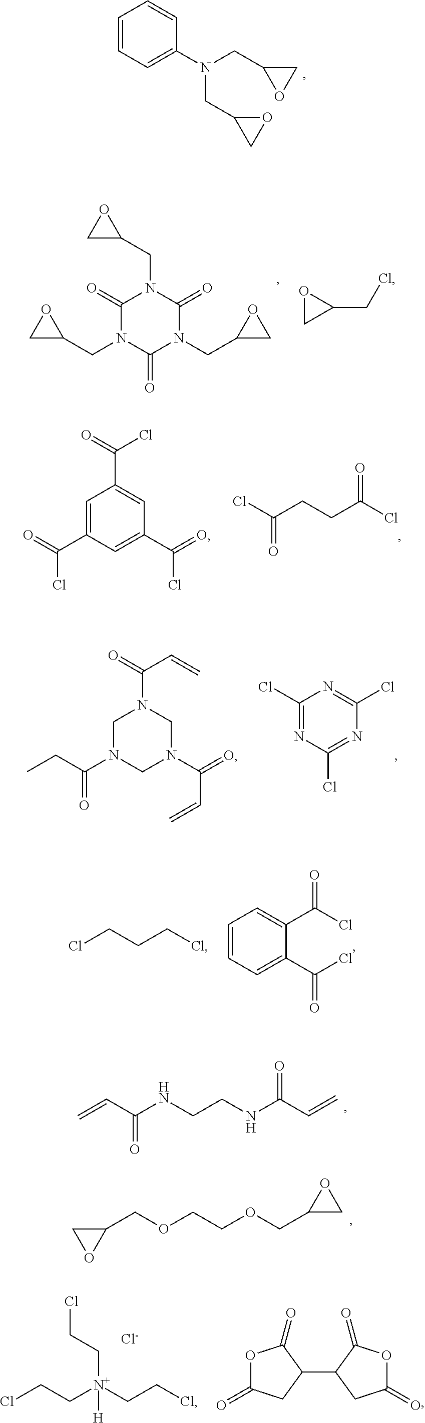

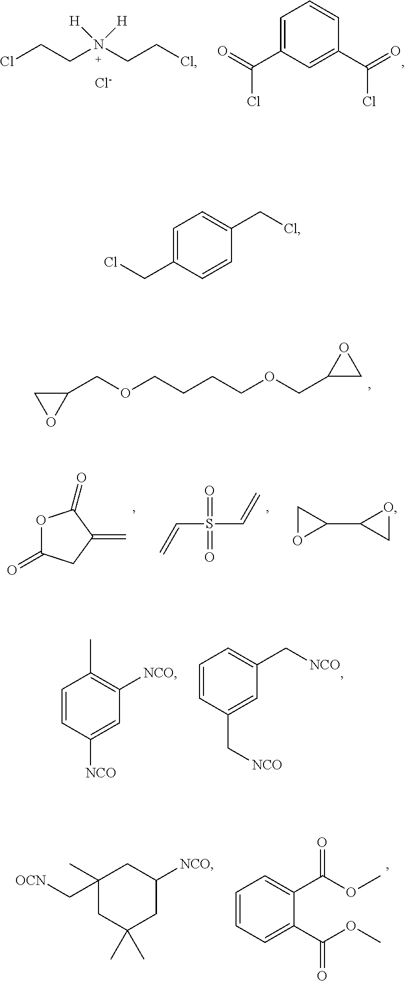

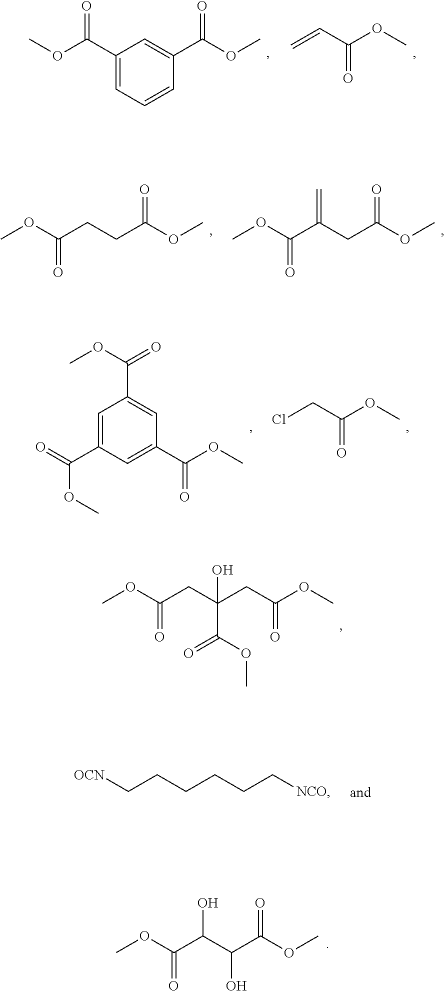

The composition wherein each of the plurality of cross-linkers is a hydrophilic cross-linker.

The composition wherein the plurality of cross-linkers comprise individual cross-linkers with a molecular weight of 200 Daltons to 6000 Daltons (Mₙ).

The composition wherein the plurality of cross-linkers comprise individual cross-linkers with a molecular weight of 400 Daltons to 2500 Daltons (Mₙ).

The composition wherein the plurality of cross-linkers comprise individual cross-linkers with a molecular weight of 800 Daltons to 2200 Daltons (Mₙ).

The composition wherein the plurality of cross-linkers are cross-linked to the polyamine polymer backbone chains at a density of about 0.01% to 10% (e.g., 0.01% to 7.5%, 0.01% to 5%, 0.01% to 2%, 0.05% to 7.5%, 0.05% to 5%, 0.05% to 2%, or 0.01%, 0.05%, 0.1%, 0.2%, 0.5%, 0.75%, 1%, 2%, 5%, 7.5%, or 10%) by molar ratio of total amines in the plurality of polyamine polymer backbone chains.

The composition wherein the plurality of cross-linkers are cross-linked to the polyamine polymer backbone chains at a density less than or equal to 1% by molar ratio of total amines in the plurality of polyamine polymer backbone chains.

The composition wherein the one or more chelators each comprise a phenyl group substituted with at least two hydroxyl groups (e.g., two hydroxyl groups), and the at least two hydroxyl groups include a vicinal diol.

The composition wherein the one or more chelator each comprises 2,3-dihydroxybenzoic acid.

The composition wherein the polymeric chelator comprises a plurality of groups each having the structure:

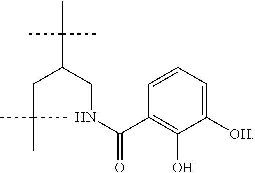

The composition wherein the one or more chelators each comprise a derivative of a metal chelator moiety.

The composition wherein the one or more chelators each comprise a derivative of deferoxamine, phytic acid, oxalic acid, polyglycerol, polyphenol, benzene-1,2-diol, benzene-1,2,3-triol, 1,10-phenanthroline, or N,N-bis(2-hydroxybenzyl)ethylenediamine-N,N-diacetic acid.

The composition wherein the one or more chelators are capable of chelating a heavy metal.

The composition wherein the one or more chelators are capable of chelating aluminum, arsenic, cadmium, chromium, copper, iron, lead, manganese, mercury, or combination thereof. The composition wherein the one or more chelators may selectively bind iron in the presence of aluminum, arsenic, cadmium, chromium, copper, lead, manganese, mercury, or a combination thereof.

The composition wherein the one or more chelators are capable of chelating iron.

The composition wherein the one or more chelators are coupled to 5-30% (e.g., 5-25%, 10-25%, 10-20%, 15-20%, 5%, 10%, 15%, 20%, 25%, or 30%) of the amines on the plurality of polyamine polymer backbone chains and the one or more chelators.

The composition wherein the polymeric chelator comprises a plurality of polymeric chelator particles each comprising the plurality of polyamine polymer backbone chains and the one or more chelators.

The composition wherein the polymeric chelator comprises a plurality of polymeric chelator particles each comprising the plurality of polyamine polymer backbone chains and the one or more chelators, and wherein at least 90% (e.g., at least 95% or at least 99%) of the plurality of polymeric chelator particles have a particle size of 300 μm or less (e.g., as measured by laser diffraction).

The composition wherein the polymeric chelator comprises a plurality of polymeric chelator particles each comprising the plurality of polyamine polymer backbone chains and the one or more chelators, and wherein at least 90% (e.g., at least 95% or at least 99%) of the plurality of polymeric chelator particles have a particle size of 2 μm to 300 μm (e.g., as measured by laser diffraction).

The composition wherein the polymeric chelator comprises a plurality of polymeric chelator particles each comprising the plurality of polyamine polymer backbone chains and the one or more chelators, and wherein at least 90% (e.g., at least 95% or at least 99%) of the plurality of polymeric chelator particles have a particle size of 4 μm to 200 μm (e.g., as measured by laser diffraction).

The composition wherein the polymeric chelator comprises a plurality of polymeric chelator particles each comprising the plurality of polyamine polymer backbone chains and the one or more chelators, and wherein at least 90% (e.g., at least 95% or at least 99%) of the plurality of polymeric chelator particles have a particle size of 4 μm to 150 μm (e.g., as measured by laser diffraction).

The composition wherein the polymeric chelator comprises a plurality of polymeric chelator particles each comprising the plurality of polyamine polymer backbone chains and the one or more chelators, and wherein at least 90% (e.g., at least 95% or at least 99%) of the plurality of polymeric chelator particles have a particle size of 5 μm to 100 μm (e.g., as measured by laser diffraction).

In an embodiment, the composition is formulated for injection.

In an embodiment, the composition is formulated for ingestion.

The composition wherein the polymeric chelator is a hydrogel.

In another aspect, provided is a method comprising administering to a subject the composition described above, including any of the embodiments described herein, alone or in any combination.

In another aspect, provided is a method for removing a metal from a medium containing the metal, the method comprising (A) applying to the medium (1) a polymeric chelator comprising a plurality of polyamine polymer backbone chains and one or more chelators; and (2) an agent selected from an antacid, a histamine H2-receptor antagonist, a proton pump inhibitor, and a combination thereof, wherein the one or more chelators are covalently coupled to one or more primary and/or secondary amines (e.g., one or more primary amines) of at least one of the plurality of polyamine polymer backbone chains, and wherein the plurality of polyamine polymer backbone chains are cross-linked with a plurality of cross-linkers; (B) incubating the polymeric chelator and the agent in the medium containing the metal to form a polymeric chelator-metal complex; and (C) removing the polymeric chelator-metal complex from the medium. Embodiments of the method for removing a metal from a medium containing the metal include the following, alone or in any combination.

The method for removing a metal from a medium containing the metal wherein the polymeric chelator and the agent are separately applied to the medium.

The method for removing a metal from a medium containing the metal wherein the polymeric chelator and the agent are formulated as the composition described above, including any of the embodiments described herein, alone or in any combination.

In another aspect, provided is a method for treating iron overload disease in a subject, the method comprising administering to the subject an effective amount of (1) a polymeric chelator comprising a plurality of polyamine polymer backbone chains and one or more chelators; and (2) an agent selected from an antacid, a histamine H2-receptor antagonist, a proton pump inhibitor, and a combination thereof, wherein the one or more chelators are covalently coupled to one or more primary and/or secondary amines (e.g., one or more primary amines) of at least one of the plurality of polyamine polymer backbone chains, and wherein the plurality of polyamine polymer backbone chains are cross-linked with a plurality of cross-linkers. As used herein, and as well understood in the art, “to treat” a condition or “treatment” of various diseases and disorders is an approach for obtaining beneficial or desired results, such as clinical results. Beneficial or desired results can include, but are not limited to, alleviation of one or more symptoms or conditions; diminishment of extent of disease, disorder, or condition; stabilizing (i.e., not worsening) state of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment. The term “effective amount,” as used herein, refers to an amount sufficient to effect beneficial or desired results, such as clinical results, and, as such, an “effective amount” depends upon the context in which it is being applied. The term “subject,” as used herein, can be a human, non-human primate, or other mammal, such as but not limited to dog, cat, horse, cow, pig, goat, monkey, rat, mouse, and sheep. In some embodiments, the subject is a human. Embodiments of the method of treating iron overload disease include the following, alone or in any combination.

The method of treating iron overload disease wherein the polymeric chelator and the agent are separately administered.

The method of treating iron overload disease wherein the polymeric chelator and the agent are formulated as the composition described above, including any of the embodiments described herein, alone or in any combination.

### BRIEF DESCRIPTION OF THE DRAWINGS

Some specific example embodiments of the disclosure may be understood by referring, in part, to the following description and the accompanying drawings.

FIGS. 1A and 1B depict structural diagrams of Enterobactin and a polymeric chelator.

FIG. 2 depicts a chart of iron binding capacity with various cross-linkers at pH 2.0 Buffer.

FIG. 3 depicts a chart of iron binding capacity with various cross-linkers at pH 3.0 Buffer.

FIG. 4 depicts a chart of iron binding capacity with various cross-linkers at pH 4.0 Buffer.

FIG. 5 depicts a chart of iron binding capacity with various cross-linkers at pH 5.0 Buffer.

FIG. 6 depicts a chart of iron binding capacity with various cross-linkers at pH 6.5 Buffer.

FIG. 7 depicts a chart showing the impact of buffer pH on iron binding capacity.

FIG. 8 depicts a chart of iron binding capacity with various cross-linking densities.

FIG. 9 depicts a chart of iron binding capacity with various cross-linking densities.

FIG. 10 depicts a chart showing the influence of pH (CaCO₃ content) on iron binding capacity of a chelator with polyethylene glycol with a molecular weight of about 1000 Daltons (PEG1000) as cross-linker.

FIG. 11 depicts a chart showing the influence of pH (NaHCO₃ content) on iron binding capacity of a chelator with PEG1000 as cross-linker.

FIG. 12 depicts a chart showing the influence of pH (NaHCO₃ content) on iron binding capacity of a chelator with BAM as cross-linker.

### DETAILED DESCRIPTION

Generally, the present disclosure includes new compositions and systems for chelation of metals. In some embodiments, the present disclosure provides a composition comprising a cross-linked polymeric chelator and an agent. In some embodiments, the agent is an agent that neutralizes or reduces stomach acid production, such as an antacid, a histamine H2-receptor, a proton pump inhibitor, or a combination. The cross-linked polymeric chelator can include a plurality of polymer backbone chains coupled with one or more metal chelators, and the polymer can be cross-linked with a plurality of cross-linkers. The system can include a plurality of polymer backbone chains and one or more metal chelators that are coupled together or otherwise linked so as to combine the properties of the polymer and the ability to chelate a metal, wherein the polymer backbone chains are crosslinked with a plurality of cross-linkers. In some embodiments, the plurality of cross-linkers comprise individual cross-linkers with a molecular weight of 200 Daltons to 6000 Daltons.

In some embodiments, the polymer backbone chain that is coupled with the one or more metal chelators may include any polyamine polymer such as polyallylamine (PAAm), poly(N-vinyl formamide) (PNVF), polyvinylamine (PVAm), poly(L-lysine) (PLL), polyethylenimine (PEI), or the like. The polymer may also include amino acids, and the polymer can include polypeptides and proteins.

In some embodiments, any polymer may be used that is capable of being coupled to a chelator, such as an iron chelator, which can be used for chelation so as to combine the properties of the polymer with the ability to chelate. The polymers can be any type of polymer that is linear, branched, or the like or a soluble polymer, a non-soluble polymer, or the like wherein the polymer is further cross-linked with a plurality of cross-linkers, e.g., a plurality of cross-linkers comprising individual cross-linkers with a molecular weight of about 200 Daltons to about 6000 Daltons. The polymers can include polyamines that have amine functional groups capable of participating in reactions with chelators. In some examples the polymer may comprise polyamine polymers such as PVAm and PAAm. PVAm and PAAm are polycation hydrogels consisting of reactive primary amine side groups for the conjugation of the chelator. In some embodiments, a cross-linked PVAm hydrogel may be synthesized by hydrolyzing a precursor polymer, PNVF, in a basic medium. In some embodiments, a cross-linked PAAm hydrogel may be synthesized by cross-linking the precursor PAAm chains with a with a plurality of cross-linkers, e.g., a plurality of cross-linkers comprising individual cross-linkers with a molecular weight of about 200 Daltons to about 6000 Daltons. Both hydrogels may demonstrate a high affinity and selectivity for iron at pHs similar to those found in the GI tract.

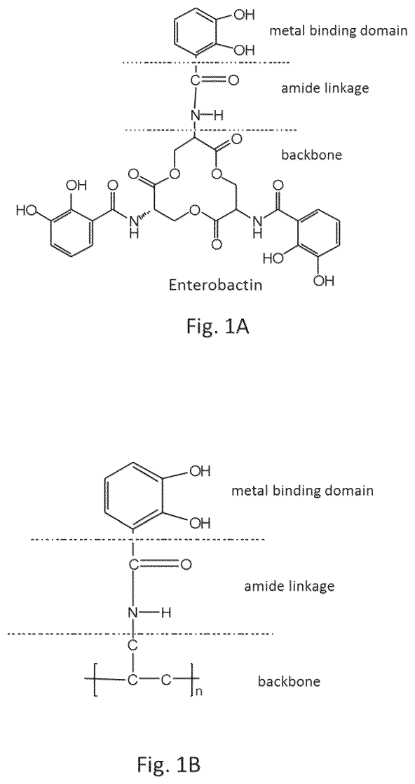

In some embodiments, the chelator coupled to the polymer may include 2,3-dihydrobenzoic acid (DHBA) and/or other iron chelators. DHBA acid is a fragment of the well-known natural iron chelator Enterobactin (Log K=52) which is a high affinity siderophore that acquires iron for microbial systems. FIG. 1A depicts a structural diagram of Enterobactin. Chelators of other metals that can be coupled to a polymer may also be included.

In some embodiments, the chelator may be coupled to the polymer via a carboxyl group of the chelator. In some embodiments, the chelator may be coupled to the polymer via a peptide bond. In some embodiments, the chelators can include a feature for coupling with the polymer, such as carboxy groups (including activated carboxy groups, e.g., N-hydroxysuccinimide (NHS)-activated carboxy groups, or activated carboxylate groups) that can be coupled to the amines of the polymer through amide bonds. Other examples of features that can be included in the chelators for coupling with the polymer include, but are not limited to, epoxide, vinyl amide, vinyl sulfonamide, anhydride, aldehyde, isocyanate, isothiocyanate, haloalkyl (e.g., chloroalkyl or bromoalkyl), haloaryl (e.g., fluorophenyl or chlorophenyl), carbonate, N-hydroxysuccinimide ester, imidoester, haloaryl ester (e.g., fluorophenyl ester), 4-nitrophenyl ester, carbodiimide, sulfonyl chloride, acyl azide, alkyl ester, vinyl acyl, succinic anhydride, and chloroacyl. In some embodiments, the feature may be any one of the following groups:

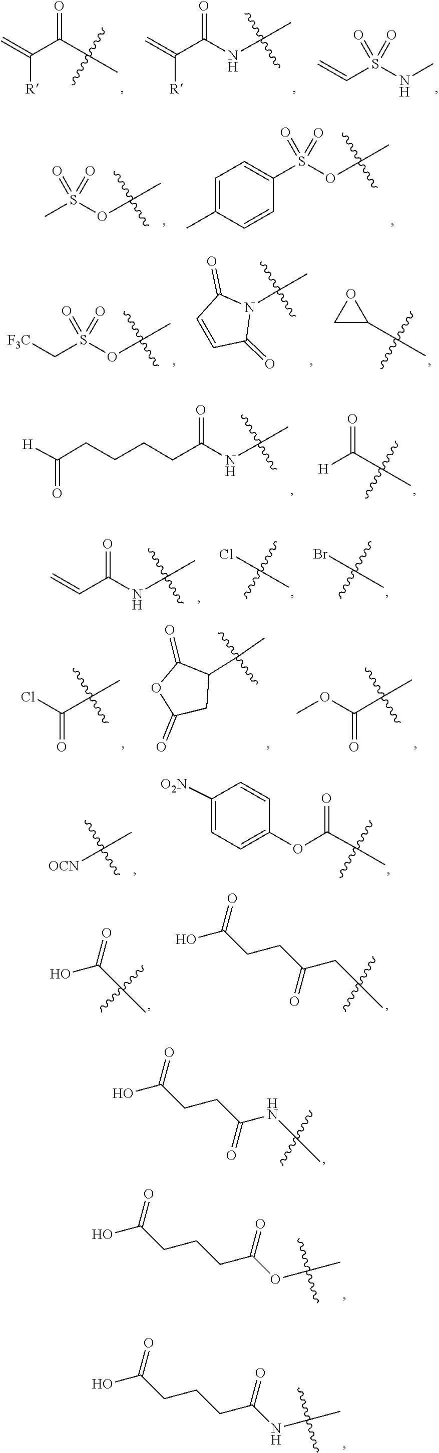

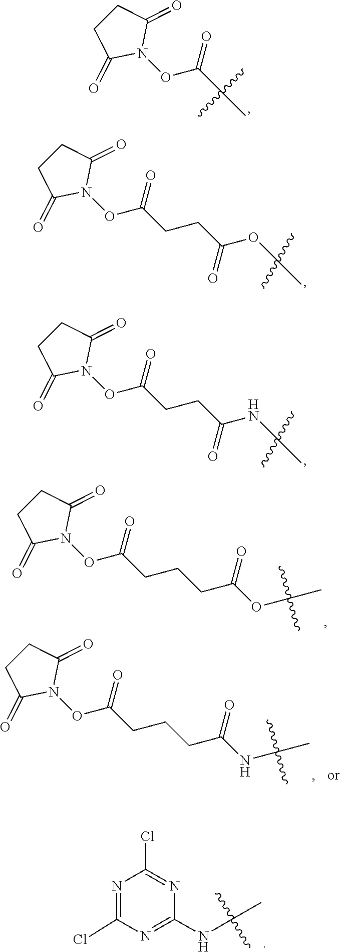 [SMILES: *C(=O)ON1C(=O)CCC1=O ; *OC(=O)CCC(=O)ON1C(=O)CCC1=O ; *NC(=O)CCC(=O)ON1C(=O)CCC1=O ; *OC(=O)CCCC(=O)ON1C(=O)CCC1=O ; *NC(=O)CCCC(=O)ON1C(=O)CCC1=O ; *Nc1nc(Cl)nc(CCl)n1]

Other coupling reagents can be included in the polymer and chelator system in order to prepare a polymeric chelator having the ability to chelate iron. Examples of iron chelating small molecules are referenced in U.S. Pat. No. 3,758,540. Examples of chelator schemes may be found in U.S. Pat. Nos. 7,342,083, 5,702,696, and 5,487,888.

Those of skill in the art will appreciate other chelators. By way of example but not limitation, additional chelators to be tested may include commercially available chelators such as Desferal® (deferoxamine mesylate) and/or may contain moieties such as phenolates, enolic hydroxyls, ketones, aldehydes, carboxylates, phosphates and phosphonates, thiolates, sulfides and disulfides, hydroxamic acids and hydroxamates, amines, amides, and nitrones. In some embodiments, the chelator may be a derivative of deferoxamine, phytic acid, oxalic acid, polyglycerol, polyphenol, benzene-1,2-diol, benzene-1,2,3-triol, 1,10-phenanthroline, or N,N-bis(2-hydroxybenzyl)ethylenediamine-N,N-diacetic acid, e.g., a derivative of the aforementioned groups that is derivatized to include any one of the features for coupling with the polymer described above.

In some embodiments, the polymeric chelator, is made by reacting 2,3-dihydroxybenzoic acid (DHBA), a known iron chelator, to a polyamine polymer. FIG. 1B depicts a structural diagram of such a polymeric chelator formed by reacting a chelator with a polyamine polymer. Notably, the polyamine polymer may be further cross-linked with a plurality of cross-linkers comprising individual cross-linkers with a molecular weight of 200 Daltons to 6000 Daltons.

In some embodiments, the composition can be fabricated as solids or equilibrated in aqueous solution as a solution or suspension. The polyamine conjugates have exceptional binding affinity and selectivity for iron. In some examples the polyamine polymer may comprise PVAm and PAAm. PVAm and PAAm are polycation hydrogels consisting of reactive primary amine side groups for the conjugation of DHBA. DHBA acid is a fraction of the well-known natural iron chelator Enterobactin (Log K=52) which is a high affinity siderophore that acquires iron for microbial systems. Cross-linked PVAm hydrogel may be synthesized by hydrolyzing a precursor polymer, PNVF, in a basic medium in the presence of a plurality of cross-linkers; e.g., individual cross-linkers with a molecular weight of 200 Daltons to 6000 Daltons. Cross-linked PAAm hydrogel may be synthesized by cross-linking the precursor PAAm chains with a plurality of cross-linkers, e.g., a plurality of cross-linkers comprising individual cross-linkers with a molecular weight of 200 Daltons to 6000 Daltons, such as 200 Daltons, 400 Daltons, 600 Daltons, 1000 Daltons, 2000 Daltons, or 6000 Daltons. Both types of polymeric chelator may demonstrate a high affinity and selectivity for iron at pHs similar to the GI tract.

In embodiments, the polyamine polymer backbone chains each comprise polyallylamine or poly(L-lysine).

In some embodiments, the composition comprises an antacid. Those of skill in the art will appreciate that antacids neutralize acidity and may comprise CaCO₃, NaHCO₃, MgCO₃, Mg(OH)₂, MgO, Al(OH)₃, simethicone, and the like, including any other antacid known by those of skill in the art. In some embodiments, the composition comprises a histamine H2-receptor antagonist, e.g., cimetidine, famotidine, nizatidine, or ranitidine. In some embodiments, the composition comprises a proton pump inhibitor, e.g., omeprazole, esomeprazole, lansoprazole, rabeprazole, pantoprazole, dexlansoprazole. In some embodiments, the composition comprises a combination of an antacid and a histamine H2-receptor antagonist. In some embodiments, the composition comprises a combination of an antacid and a proton pump inhibitor (e.g., omeprazole and NaHCO₃). In some embodiments, the composition comprises a combination of a histamine H2-receptor antagonist and a proton pump inhibitor. In some embodiments, the composition comprises a combination of an antacid, a histamine H2-receptor antagonist, and a proton pump inhibitor.

In embodiments, the plurality of cross-linkers comprise bisacrylamide units.

In embodiments, the plurality of cross-linkers each comprise polyethylene glycol. “Polyethylene glycol” or “PEG,” as used herein, refers to a group of the general formula —(OCH₂CH₂)ₙO—, in which n is an integer (e.g., 2-150, 2, 3, 4, 5, 5-10, 10-20, 20-30, 30-40, 50-60, 60-70, 70-80, 80-90, 90-100, 100-110, 110-120, 120-130, 130-140, or 140-150). In some embodiments, the PEG has a molecular weight (Mₙ) of 200 Da to 6000 Da (e.g., 400 Da to 2500 Da, 800 Da to 2200 Da, 1000 Da to 2000 Da, 200 Da, 400 Da, 600 Da, 800 Da, 1000 Da, 1200 Da, 1500 Da, 2000 Da, 2200 Da, 2500 Da, 3000 Da, 3500 Da, 4000 Da, 4500 Da, 5000 Da, 5500 Da, or 6000 Da) PEG-based crosslinkers are generally known in the art and are commercially available.

PEG-based crosslinkers for amine PEGylation include reactive end groups include, but are not limited to carboxy, epoxide, vinyl amide, vinyl sulfonamide, anhydride, aldehyde, isocyanate, isothiocyanate, haloalkyl (e.g., chloroalkyl or bromoalkyl), haloaryl (e.g., fluorophenyl or chlorophenyl), carbonate, N-hydroxysuccinimide ester, imidoester, haloaryl ester (e.g., fluorophenyl ester), 4-nitrophenyl ester, carbodiimide, sulfonyl chloride, acyl azide, alkyl ester, vinyl acyl, succinic anhydride, and chloroacyl. In embodiments, the plurality of cross-linkers are derived from polyethylene glycol diacrylate units.

In some embodiments, a PEG-based crosslinker comprises two or more PEG chains connected via one or more linkers. Molecules that may be used as linkers include at least two functional groups (which may be the same or different) that can form covalent linkages with the reactive end groups of individual PEG chains. The functional groups include, but are not limited to, amine, carboxy, epoxide, vinyl amide, vinyl sulfonamide, anhydride, aldehyde, isocyanate, isothiocyanate, haloalkyl (e.g., chloroalkyl or bromoalkyl), haloaryl (e.g., fluorophenyl or chlorophenyl), carbonate, N-hydroxysuccinimide ester, imidoester, haloaryl ester (e.g., fluorophenyl ester), 4-nitrophenyl ester, carbodiimide, sulfonyl chloride, acyl azide, alkyl ester, vinyl, vinyl acyl, succinic anhydride, and chloroacyl. In some embodiments, the individual PEG chains each include two different reactive end groups, e.g., one for forming a conjugate linkage with the linker, and one for forming a conjugate linkage with an amine on a polyamine polymer backbone chain. Strategies for forming linkages between individual PEG chains are generally known in the art.

In embodiments, the plurality of cross-linkers are derived from polyethylene glycol diglycidyl ether units.

In embodiments, the plurality of cross-linkers comprise individual hydrophilic cross-linkers. In some embodiments, the hydrophilic cross-linker is a compound having a water solubility greater than that of N,N′-methylene bisacrylamide at 20° C. In some embodiments, the hydrophilic cross-linker is a compound having a water solubility of greater than 20 g/L (e.g., at least 50 g/L, at least 100 g/L, at least 150 g/L, at least 200 g/L, at least 250 g/L, at least 300 g/L, at least 500 g/L, at least 550 g/L, at least 600 g/L, or at least 650 g/L) at 20° C.

In embodiments, the plurality of cross-linkers comprise individual cross-linkers preferably have a molecular weight of 400 Daltons to 2500 Daltons, or 400 to 1200 Daltons, or 400 to 1000 Daltons, more preferably having a molecular weight of 800 Daltons to 2200 Daltons, or 800 Daltons to about 2000 Daltons.

The composition wherein the plurality of cross-linkers are cross-linked to the polymer chains at a density of 0.01% to 10% (e.g., 0.01% to 7.5%, 0.01% to 5%, 0.01% to 2%, 0.05% to 7.5%, 0.05% to 5%, 0.05% to 2%, or 0.01%, 0.05%, 0.1%, 0.2%, 0.5%, 0.75%, 1%, 2%, 5%, 7.5%, or 10%) by molar ratio of total amines, preferably at a density less than or equal to 1% by molar ratio of total amines, such as 0.01% to 1% by molar ratio of total amines.

Of note are cross-linked polymers wherein the crosslinkers have a molecular weight of 400 Daltons to 1200 Daltons and a cross-linking density of 0.010% to 2% by molar ratio of total amines; cross-linked polymers wherein the crosslinkers have a molecular weight of 400 Daltons to 1200 Daltons and a cross-linking density of 0.01% to 5% by molar ratio of total amines; cross-linked polymers wherein the crosslinkers have a molecular weight of 800 Daltons to 2200 Daltons and a cross-linking density of 0.01% to 2% by molar ratio of total amines; cross-linked polymers wherein the crosslinkers have a molecular weight of 800 Daltons to 2200 Daltons and a cross-linking density of 0.01% to 5% by molar ratio of total amines.

In some embodiments, conjugation of DHBA may facilitate the iron binding affinity and iron selectivity of the final hydrogel conjugates, the cross-linked polymeric chelator. In some embodiments, the primary amine groups in both polymers may be used as a conjugation site. The non-degradable PVAm and PAAm hydrogels conjugated to DHBA can be used as oral therapeutics in iron overload disease patients. This therapeutic agent can selectively bind iron and remove it from the GI tract before it is being absorbed into the blood stream.

In some embodiments, the present disclosure provides a composition comprising a plurality of polymeric chelator particles in which at least 90% of the particles have a particle size of 300 μm or less, such as 300 μm to 2 μm, 200 μm to 4 μm, 150 μm to 4 μm, 100 μm to 5 μm, or 70 μm to 18 μm, 45 μm to 18 μm, or 45 μm or less, such as a particle size of 300 μm, 275 μm, 250 μm, 225 μm, 200 μm, 175 μm, 150 μm, 125 μm, 100 μm, 75 μm, 65 μm, 55 μm, 50 μm, 45 μm, 40 μm, 35 μm, 30 μm, 25 μm, 20 μm, 18 μm, 15 μm, 12.5 μm, 10 μm, 7.5 μm, 5 μm, 4 μm, 3 μm, 2 μm, or 1 μm. In some embodiments, the size of the polymeric chelator particles is determined using laser diffraction, for example by using a Malvern Masterizer. In embodiments, the size of the polymeric chelator particles measured by laser diffraction comprises a d10 of 100 μm or less, such as a d10 of 100 μm, 75 μm, 65 μm, 55 μm, 50 μm, 45 μm, 40 μm, 35 μm, 30 μm, 25 μm, 20 μm, 18 μm, 15 μm, 12.5 μm, 10 μm, 7.5 μm, 5 μm, 4 μm, 3 μm, 2 μm, or 1 μm. In embodiments, the size of the polymeric chelator particles measured by laser diffraction comprises a d50 of 150 μm or less, such as a d50 of 150 μm, 125 μm, 100 μm, 75 μm, 65 μm, 55 μm, 50 μm, 45 μm, 40 μm, 35 μm, 30 μm, 25 μm, 20 μm, 18 μm, 15 μm, 12.5 μm, 10 μm, 7.5 μm, 5 μm, 4 μm, 3 μm, 2 μm, or 1 μm. In embodiments, the size of the polymeric chelator particles measured by laser diffraction comprises a d90 of 250 μm or less, such as a d90 of 250 μm, 225 μm, 200 μm, 175 μm, 150 μm, 125 μm, 100 μm, 75 μm, 65 μm, 55 μm, 50 μm, 45 μm, 40 μm, 35 μm, 30 μm, 25 μm, 20 μm, 18 μm, 15 μm, 12.5 μm, 10 μm, 7.5 μm, 5 μm, 4 μm, 3 μm, 2 μm, or 1 μm. In other embodiments, thioglycolic acids (TGA) in combination with the siderophore moiety DHBA may be introduced onto PAAm and PVAm to form the polymeric chelator.

In one aspect, the present disclosure provides a composition comprising a monomer having the DHBA coupled thereto. The monomer can be coupled to the DHBA by the monomer having an amine group which reacts and couples with the carboxyl group of the DHBA. The monomer having the DHBA can be used in compositions similarly to that which is described in connection with the polymer coupled to DHBA. Examples of suitable monomers include any monomer that is capable of being coupled to a chelator, such as an iron chelator. The monomer can be any type of monomer. The monomer can include amines that have amine functional groups capable of participating in reactions with chelators. In some examples the monomer may comprise amine monomers.

In some embodiments, a polymeric chelator can be made by reacting DHBA to a polyamine polymer through the formation of an amide bond and further cross-linked. The polyamine-DHBA chelating polymer has exceptional binding affinity and selectivity for iron.

In some embodiments, the polyamine polymer is PVAm or PAAm. Both PVAm and PAAm are polycation hydrogels that have reactive primary amine side groups that can be coupled to 2,3-DHBA. Cross-linked PVAm hydrogel can be synthesized by hydrolyzing a precursor polymer, PNVF, in a basic medium. Cross-linked PAAm hydrogel can be synthesized by cross-linking the precursor PAAm chains.

In some embodiments, synthesized cross-linked polymers may be washed according to a washing procedure. The washing procedure may include administering one or more washing solutions. In some embodiments, a washing solution has one or more bases. The one or more bases may be capable of quenching the synthetic reaction. In some embodiments, the one or more bases may include sodium hydroxide, potassium hydroxide, calcium hydroxide, or the like. In some embodiments, the washing solution may have a concentration of 0.01-1.0 M base in an aqueous solution (e.g., 0.01 M, 0.05 M, 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M, or 0.1 M). Alternatively, in some embodiments the washing solution may be deionized water.

In some embodiments, the washing procedure may include washing the synthesized cross-linked polymers with a first washing solution having a base, and subsequently washed with a second washing solution of deionized water. In some embodiments, the first washing solution or second washing solution may be administered under the protection of an inert gas, e.g., nitrogen, argon, helium, or the like. For example, the first washing solution or second washing solution may be administered under the protection of nitrogen gas.

The composition can be fabricated as solids, gels, pastes, or liquids, such as being equilibrated in aqueous solution as a solution or suspension.

In some embodiments, the composition can be administered orally to treat, inhibit, or prevent iron overload. As such, the composition can be included in oral therapeutics for use in iron overload disease patients. The composition can selectively bind iron and remove it from the GI tract before it is being absorbed into the blood stream. The composition can be deposited in tissues or administered systemically for iron chelation.

The composition may be used as metal chelators to remove metals from a wide range of substance and can have applications in a wide range of diverse fields. Polycations have been employed in industrial applications such as water treatment and ion exchange resins (for separation-purification purposes). The high affinity and selectivity for iron provides important features for the application of these gels.

The cross-linked polymeric chelators can be highly effective metal (e.g., iron) chelators that selectively bind metals in the GI tract and prevent the metal from being absorbed into the blood stream. The chelated metal can be passed from the GI tract as waste.

In some embodiments, the present disclosure provides a composition (e.g., any one of the compositions disclosed herein) that may be injected or ingested. In some embodiments, dosage form design may aid patient compliance. The gel format may retain chelators in the gastrointestinal tract to enable self-dosing of the compound as necessary and to mitigate systemic side effects that plague current iron chelators. The injectable composition may improve safety compared to DFO and the polymer molecular weight, including the molecular weight and cross-linking density of the cross-linkers, may be optimized to extend circulation half-life.

In one embodiment, the polymeric chelator can be configured to include a polymer or monomer that is soluble in water. The composition can be configured to be injected and to be relatively non-toxic or have reduced toxicity. In one embodiment, the polymeric chelator can be configured to have an appropriate molecular weight for injection. In another embodiment, the polymeric chelator can be configured to have an appropriate molecular weight for ingestion. Also, the composition having a polymeric chelator can be configured for inhalation or for topical application.

In one embodiment, the composition can be ingested and can block metal absorption by chelating the metal. The composition can include a cross-linked polymer configured for ingestion. In some embodiments, the composition can be ingested and be configured to be absorbed from the intestine such that the chelator can chelate metals that have already been absorbed into the body.

In some embodiments, the polymeric chelator disclosed herein may more accurately mimic the Enterobactin side chain shown. Cross-linked polymeric chelators that mimic the structure of siderophore may be considered as a desirable parenterally administered iron chelator. The plasma half-life of these polymeric agents can be optimized based on the initial molecular weight of the polymer. Moreover, the toxic side effect of these polymeric chelators may be significantly reduced because they consist of polypeptide units. Polymeric forms of siderophore mimetics offer several therapeutic advantages. These compounds can disable bacterial recruitment of iron. Also, cross-linked polymeric chelators can localize the compounds to the GI tract (oral gel material) and/or extend the circulation half-life by increasing molecular weight (injected material).

In certain embodiments, the crosslinked forms of the polymeric chelator will not be absorbed when orally given. These materials may demonstrate rapid iron binding with high affinity and selectivity. In some embodiments, the pM values for iron binding the materials disclosed herein are at least ten times higher than any of the existing therapeutic chelators. The design of these polymers can mitigate the systemic side effects and toxicity of current drugs. In some embodiments, the polymeric chelator selectively and effectively binds iron in the GI tract if administered orally or from the bloodstream if administered parenterally.

In one embodiment, the cross-linked polymeric chelators can be incorporated into textiles, fabrics, absorbent members, gauze, wipes, bandages, or the like. Further applications of the cross-linked polymeric chelators can be used for metal chelation in a wide range of consumer products and processes. An example of one process that the polymeric chelator can be useful is in oil well treatments, such as those treatments for descaling or inhibiting the formation of scales.

To facilitate a better understanding of the present invention, the following examples of specific embodiments are given. In no way should the following examples be read to limit or define the entire scope of the invention.

### EXAMPLES

### Synthesis and Characterization.

Poly(allylamine hydrochloride) (PAAm) with an average molecular weight of 15 kDa and analytical grade reagent N,N′-methylenebisacrylamide (BAM) were obtained from Sigma-Aldrich and used without further modification. 2,3-dihydroxybenzoic acid, N,N,N-triethylamine, dimethylformamide (DMF), potassium phosphate and all metal chlorides were purchased from Fisher Scientific and used as received. N-(3-Dimethylaminopropyl)-N′-ethylcarbodiimide hydrochloride (EDC) and N-hydroxysuccinimide (NHS) were purchased from Thermo Scientific and used without further modification. Polyethylene glycol diglycidyl ether were purchased from Polysciences, Inc. and used as received. Deionized water (DI) was obtained from a Barnstead EasyPure water purifier.

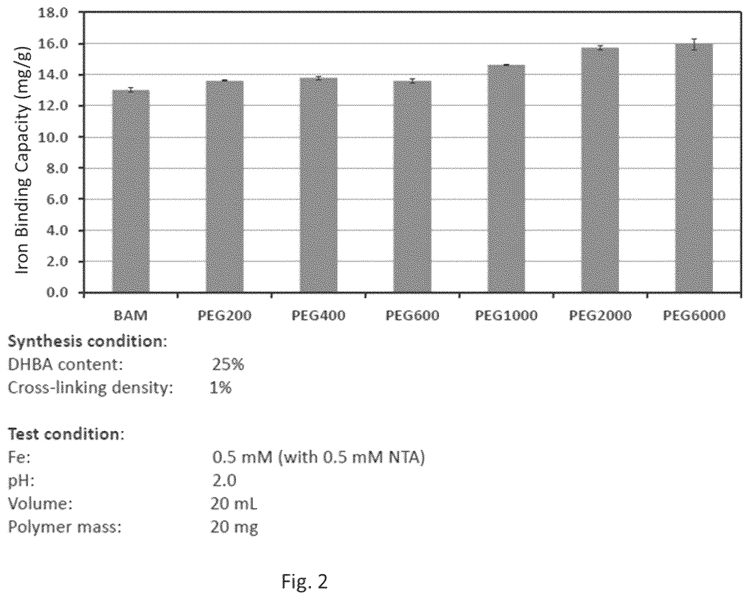

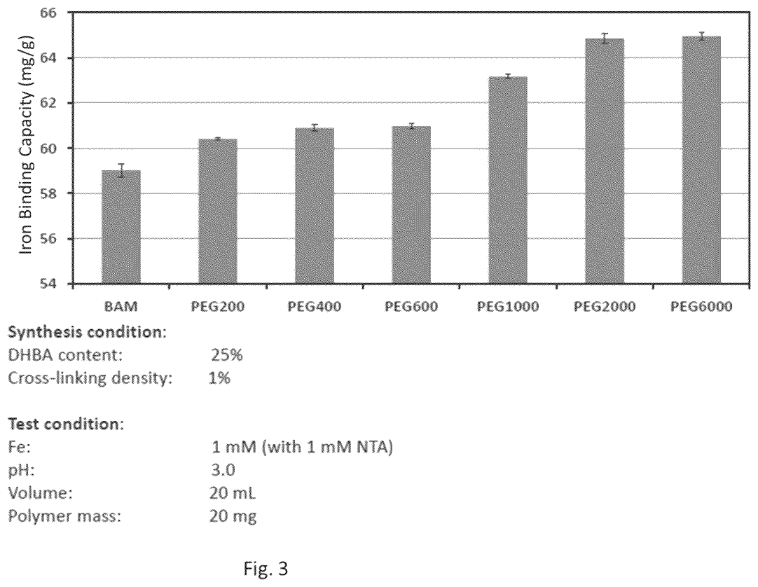

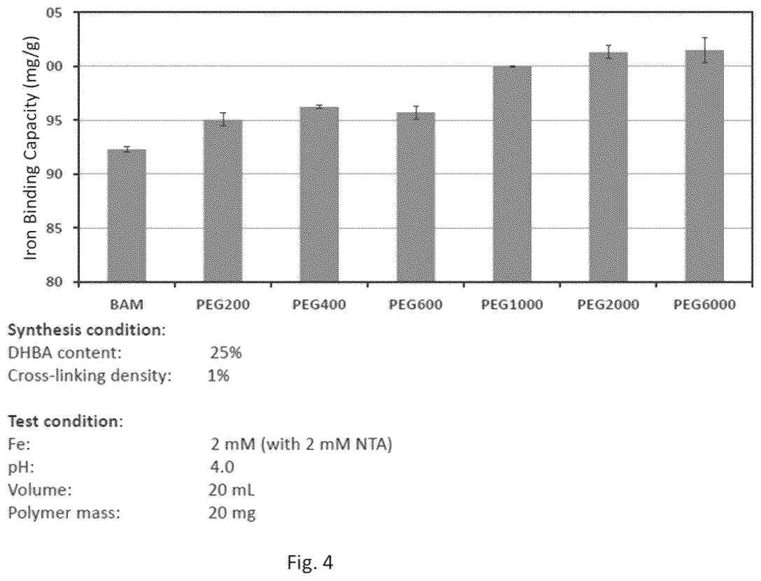

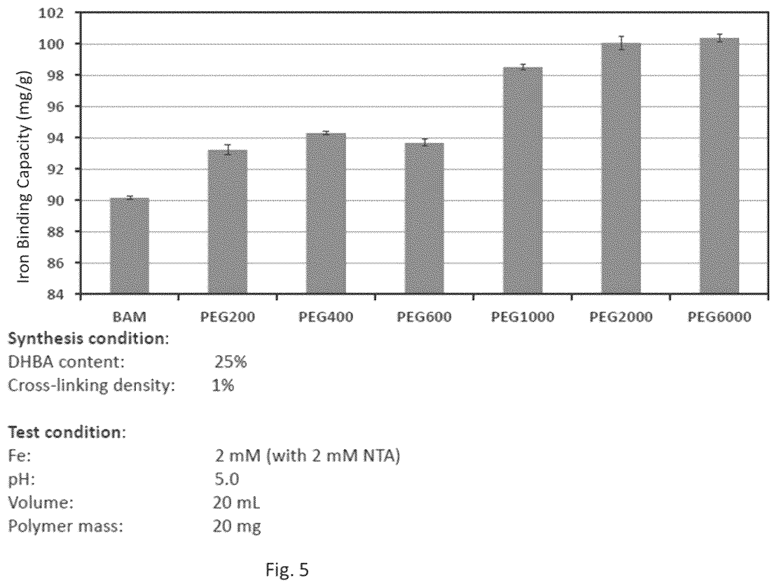

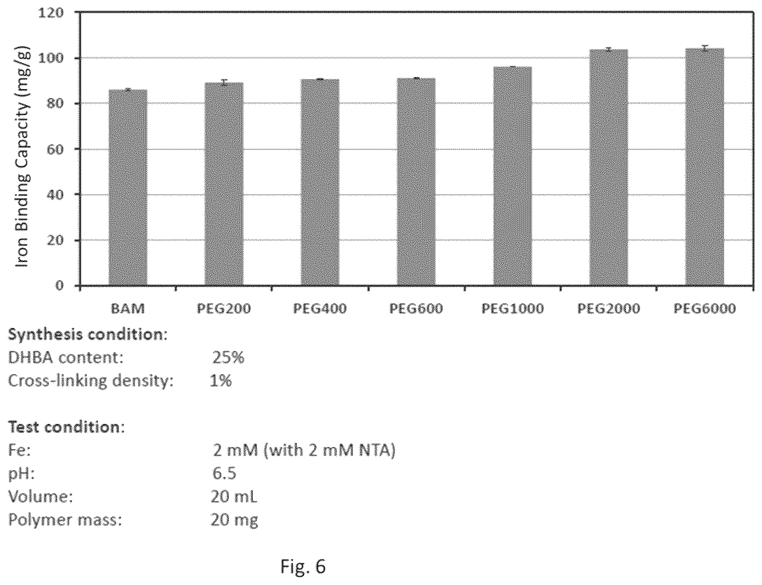

Poly(allylamine hydrochloride) was cross-linked with several polyethylene glycol diglycidyl ether units of different average molecular weight. The crosslinkers had average molecular weights of 200, 400, 600, 1000, 2000 and 6000 Daltons (See FIGS. 2-6).

The reaction was performed in water to provide a cross-linking density of 1.0% by molar ratio of total amine groups. Although cross-linking was done by linking the primary amine groups, there were still a considerable number of reactive amino sites available for further modification of the PAAm hydrogel. DHBA was covalently linked to the PAAm hydrogel via EDC/NHS conjugation chemistry.

### NHS-Activated DHBA.

A solution of DHBA (770 mg, 5 mmol) and NHS (690 mg, 6 mmol) in 5 mL of DMF was mixed with a solution of EDC (1200 mg, 6.2 mmol) in 5 mL of DMF. The mixture was stirred at room temperature for 8 h and used for the next reaction step without any purification.

### Preparation of DHBA Modification of Hydrogel.

The PAAm cross-linking and DHBA conjugation were conducted in a single-step reaction. Briefly, a 15% w/w PAAm (15 kDa) solution containing a predetermined amount of BMA (1% molar ratio of total amines) was prepared in H2O/DMF (50/50 v/v) mixture. Then, the NHS-activated DHBA solution was added to the reaction mixture, with a final DHBA/amine molar ratio of 25% and was sonicated until a transparent solution was achieved (˜2 minutes). Triethylamine (TEA) was then added to the solution and mixed thoroughly, and the solution was incubated at room temperature for 48 h. The resultant cross-linked polymers were then washed with 0.1 M sodium hydroxide and subsequently washed with deionized water for several days under the protection of nitrogen. The DHBA modified PAAm hydrogel was lyophilized and ground to fine powder by a mortar and pestle set for 5 min.

The iron binding capacity can be determined at different pH values and different isotherm models can be used to fit the data.

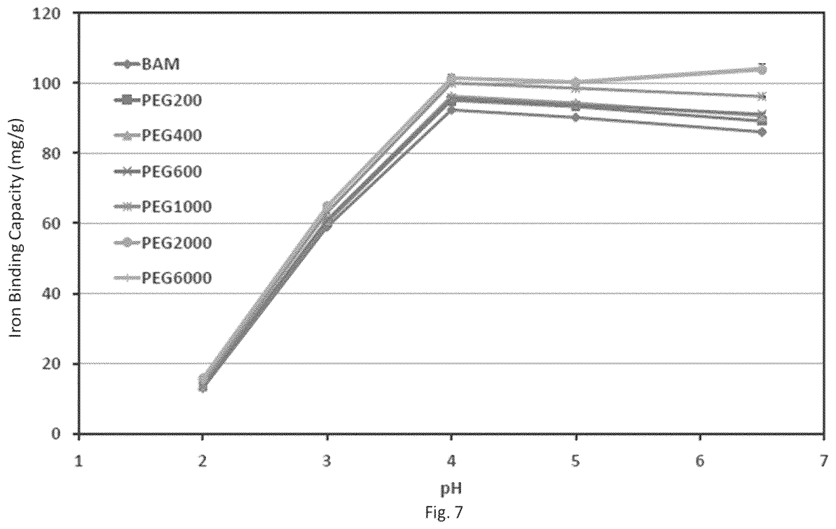

The results of iron binding capacity at pH of 2.0, 3.0, 4.0, 5.0 and 6.5 are summarized in FIGS. 2-6 respectively and compared to a polymer cross-linked with BAM. At low pH, the metal ion uptake was relatively low. Increasing the pH increased the values of the metal ion uptake. At pH of 2.0, all cross-linked polymers showed similar iron-binding capacity of about 16 to 16 mg/g. Iron-binding capacity increased at pH 3.0 and 4.0, above which the iron-binding capacity plateaued at about 90 to 100 mg/g. In general as shown in FIG. 7, binding capacity increased as cross-linker average molecular weight, and hence cross-linker length, increased. They demonstrated an almost instant iron absorbance when equilibrated in ferric solution. The cross-linked chelating polymers were hydrogels that demonstrated a high affinity and selectivity for iron at pHs similar to the GI tract and the time required for equilibration of swelling response of the polymer to gel varied in different pHs.

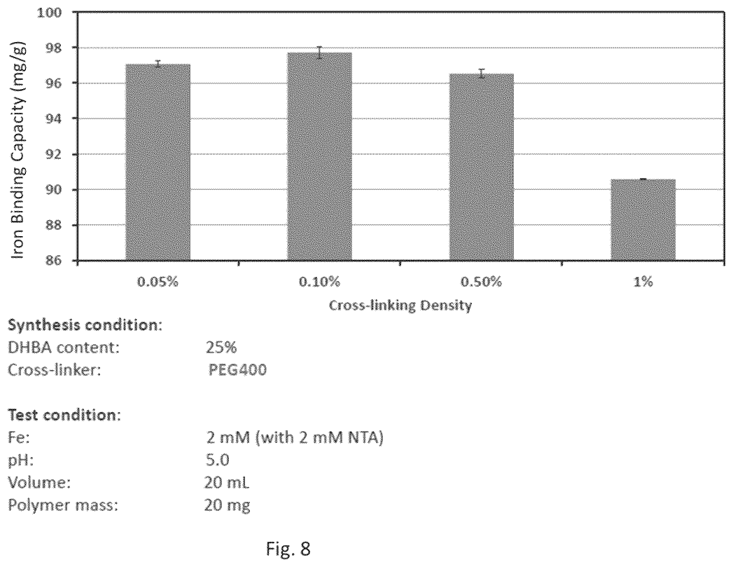

FIG. 8 shows the iron binding capacity of crosslinked polymers comprising cross-linkers having average molecular weight of about 400 Daltons with cross-linking densities of about 0.05 to about 1% at pH of 5.0. All of these crosslinked polymers exhibited iron binding capacity between 90 and 98 mg/g at a pH of 5.0. Polymers with cross-linking density of 0.05 to 0.5% exhibited iron binding capacity above 96 mg/g. The drop-off in iron binding capacity exhibited by the polymer with cross-linking density of 1% suggests that shorter cross-linkers desirably have lower cross-linking densities to provide better iron binding.

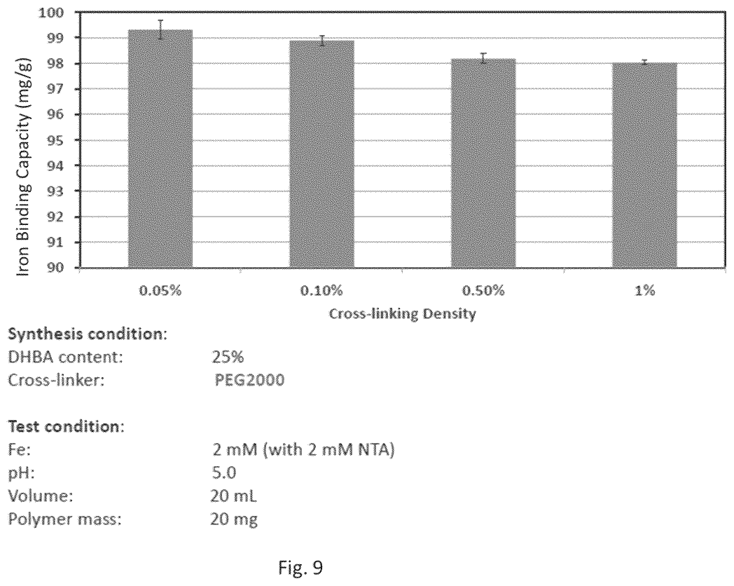

FIG. 9 shows the iron binding capacity of crosslinked polymers comprising cross-linkers having average molecular weight of about 2000 Daltons with cross-linking densities of about 0.05 to about 1% at pH of 5.0. All of these crosslinked polymers exhibited iron binding capacity of about 98 to above 99 mg/g at a pH of 5.0. The drop-off in iron binding capacity exhibited by the polymer with cross-linking density of 1% is less severe with longer cross-linkers.

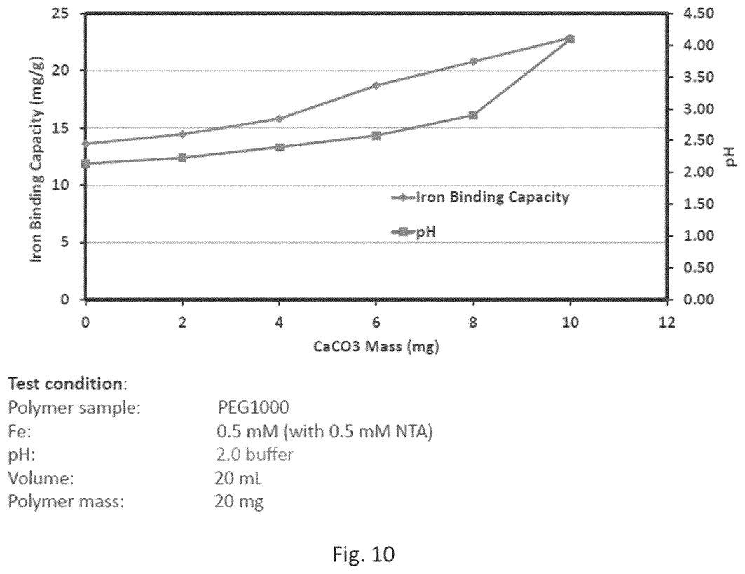

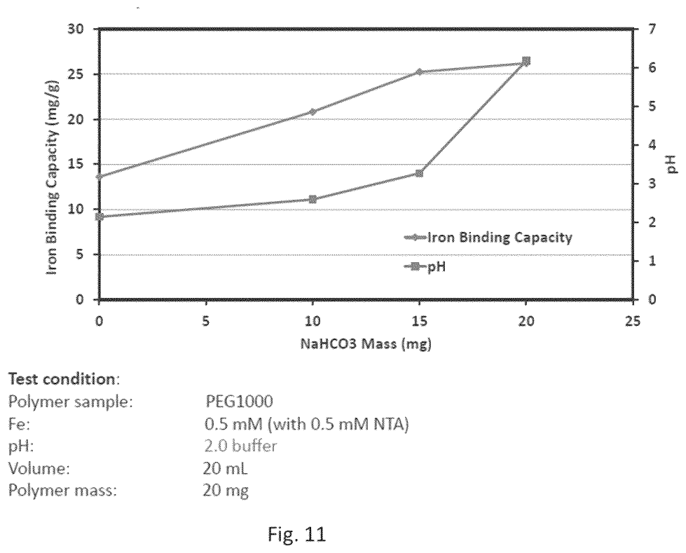

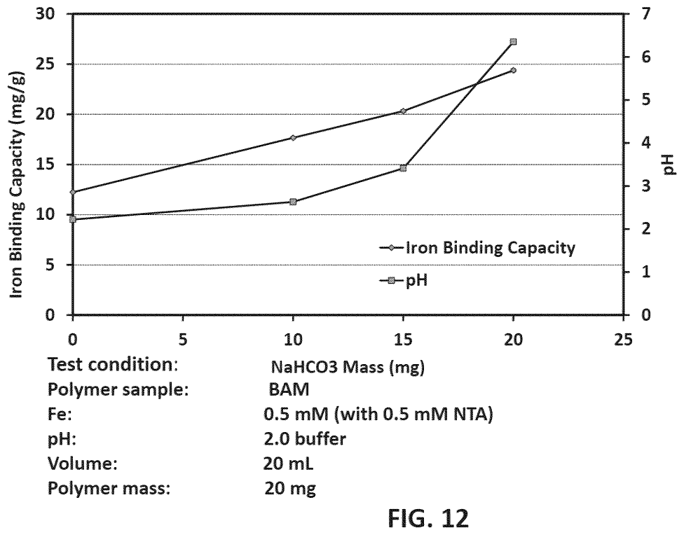

FIGS. 10-12 show the influence of added antacids such as calcium carbonate or sodium bicarbonate on iron binding capacity. FIGS. 10 and 11 depict the iron binding capacity of a chelator with a PEG1000 cross-linker with added calcium carbonate (FIG. 10 or sodium bicarbonate (FIG. 11). FIG. 12 depicts the iron binding capacity of a chelator with a bisacrylamide (BAM) cross-linker with added sodium bicarbonate. When tested at a pH buffer at 2.0 (similar to the pH of gastric fluids), with increasing levels of antacid, the iron binding capacity improved as more antacid was used. This suggests that formulating or administering the cross-linked polymers with an antacid is advantageous for improved iron binding capacity.

Both orally and parenterally administered iron chelator agents must have high selectivity and affinity for iron. Moreover, parenterally administered iron chelator agents should be non-toxic with a relatively long plasma half-life.

The respective isotherm curves of ferric and ferrous solutions can be obtained at different pH values and fit using well known models of solute absorption; Freundlich, Langmuir or Temkin.

Different isotherm models were employed to determine how the metal molecules distributed between the liquid phase and the solid hydrogel phase when the adsorption process reached equilibrium state. Langmuir, Freundlich, and Temkin isotherm models were applied to the data. Adsorption parameters of ferric and ferrous ions were calculated at different pHs. The accuracy of the isotherm models can be evaluated by linear correlation coefficient (R²) values.

### Langmuir Isotherm.

Langmuir isotherms assume monolayer adsorption onto a surface containing a finite number of adsorption sites. The linear form of the Langmuir isotherm equation is given as:

$$\frac{{C}_{e}}{{q}_{e}} = \frac{1}{{q}_{m ax} {K}_{L}} + \frac{{C}_{e}}{{q}_{m ax}}$$

where Cₑ is the equilibrium concentration of the metal ion (mg/L), qₑ is the amount of metal ion adsorbed per unit mass of hydrogel (mg/g), K_{L} and qₘₐₓ are Langmuir constants related to the adsorption/desorption energy and adsorption capacity, respectively. When Cₑ/qₑ was plotted against Cₑ, a straight line with slope of 1/qₘₐₓ was obtained. The Langmuir constants K_{L} and qₘₐₓ are calculated from Eq. (2).

### Freundlich Isotherm.

Freundlich isotherms assume heterogeneous surface energies, in which the energy term in the Langmuir equation varies as a function of the surface coverage. The linear form of the Freundlich isotherm is given by the following equation:

$$ln {q}_{e} = ln {K}_{F} + \frac{1}{n} ln {C}_{e}$$

where Cₑ is the equilibrium concentration of the metal ion (mg/L), qₑ is the amount of metal ion adsorbed per unit mass of hydrogel (mg/g), K_{F} (mg/g (l/mg)l/n) and n are Freundlich constants with n giving an indication of how favorable the absorption process is. The plot of lnqₑ versus lnCₑ gives a straight line with slope of l/n. Freundlich constants K_{F} and n also can be calculated.

### Temkin Isotherm.

Temkin and Pyzhev considered the effects of indirect adsorbate/adsorbent interactions on adsorption isotherms. The heat of adsorption of all the molecules in the layer would decrease linearly with coverage due to adsorbate/adsorbent interactions. The Temkin isotherm has been used in the form as follows:

$${q}_{e} = ( \frac{RT}{b} ) ln ( A {C}_{e} )$$

A plot of qₑ versus lnCₑ yields a straight line.

### Swelling Studies.

The swelling kinetics of PAAm and PAAm/DHBA can be studied to determine the time to reach equilibrium. Hydrogel swelling increased with time; however, it eventually plateaus, thus, allowing calculation of the equilibrium swelling percentage. The cross-linker concentration is varied and the swelling behaviors of the hydrogels are determined.

High throughput cell viability assays may be completed using standard procedures. Cytotoxicity of polymers may be determined by the CellTiter 96® Aqueous Cell Proliferation Assay (Promega). HUVEC cells may be cultured and incubated with polymers for ˜24 h. The media may then be removed and replaced with a mixture of 100 μL fresh culture media and 20 μL MTS reagent solution. The cells may be incubated for 3 hours at 37° C. in a 5% CO₂ incubator. The absorbance of each well may then be measured at 490 nm using a microtiter plate reader (SpectraMax, M25, Molecular Devices Corp.) to determine relative cell viability. A similar study may be conducted on polymer gels for oral delivery using Caco-2 cells (colon epithelium).

Female Sprague-Dawley rats, ˜6 weeks old may be used to assess treatment effects on iron load. The initial iron level in the blood of rats may be measured before starting the experiment after animals have equilibrated to diet and environment at KU.

The animals may be fed 25 mg/kg of the gel-containing diet for 4 days, which may provide adequate time to allow clearance of untreated intestinal contents. Urine, fecal, and blood samples may then be collected from each animal on days 5 and 10. Animals receiving the injected chelators Deferoxamine (DFO), SiMiP-01 or SiMiP-02 may be treated with subcutaneous injections of 40 mg/kg every 2 days during a 10-day period (e.g. injections on day 2, 4, 6, etc). High molecular weight polymer (>25 kDa) may not be well absorbed; therefore, tail vein injections may be used in lieu of subcutaneous injections if larger polymers are identified as better iron chelators. The iron level in the blood, feces, and urine may be measured using ICP-MS. Animals may be continually monitored for distress and blood samples from animals receiving injected chelators may be analyzed for aspartate aminotransferase, alanine transamidase, total bilirubin, alkaline phosphatase and/or urea nitrogen and serum creatinine to monitor liver and kidney function, respectively. The experimental protocol may involve healthy animals and moderate doses of iron; therefore, animal health is not expected to be compromised. SiMiG-03 and SiMiP-03 may also be tested if another suitable polymer is identified.

### Quantification of Amine Functional Groups.

Primary amine groups may be quantified by potentiometric titration. After grinding to a powder, 40 mg of cross-linked polymer are suspended in 35 mL of 0.2 M aqueous KCl solution. Next, 140 μL of 8 M KOH aqueous solution is added to polymer suspensions to raise the pH to ˜12. Standard 0.1 M HCl is used to titrate the suspension. HCl is added until the pH is about 2.5 in the polymer suspensions. Free amine groups are quantified from potentiometric data following reported procedures.

### Polymer-Iron Stability Constant Determination.

The stability constant or ‘binding coefficient’ of gel chelators may be measured using an historic ligand competition assay. Competitive chelation of iron by cross-linked polymeric chelators in equilibrium with a water-soluble chelator (ethylenediaminetetraacetic acid:EDTA) may be used to determine the stability constant of iron-ligand complexes of the polymers. Briefly, to a 1.5 ml of 10 mM EDTA solution may be added 2 mL of 5 mM of FeCl₃ solution and 21.5 mL PBS and a known mass of gel. The mixture may be rotated at 20° C. for 3 days and the concentration of the soluble iron complex may be determined by inductively coupled plasma mass spectroscopy (ICP-MS). The stability constant of the gel may be determined following the procedure reported in literature. Stability constants may also be determined by means of potentiometric titration to confirm results.

### Selectivity Study.

The selectivity for Fe by PAAm-DHBA in the presence of several heavy metals such as copper, zinc, manganese, calcium, and potassium can be studied. Metal solution (10 mL) containing all metal components is prepared. The upper tolerable intake level of each metal is used as an initial concentration in the solution. These concentrations are chosen on the basis of the U.S. recommended daily allowance (RDA) data on the daily dietary uptake of these metal ions present in a normal meal. The solution mixture is then adjusted to pH 2.5 and held at room temperature for 2 hours after adding a known mass of cross-linked polymeric chelators as a dry gel.

### Metal Analysis.

Mono- and multi-elemental analysis of samples can be quantified by Inductively Coupled Plasma Optical Emission Spectrometry (ICP-OES) (Optima 2000 DV, PerkinElmer, USA) fitted with an AS 93plus autosampler (PerkinElmer, USA). A Cross-Flow nebulizer and a Scott spray chamber are used. The RF Power is 1300 W and nebulizer and auxiliary flows are 0.8 and 0.2 L/min, respectively. Sample flow is set at 1.5 mL/min. ICP-OES data is processed using Winlab 32 (Ver. 3.0, PerkinElmer, USA).

The EDC/NHS coupling chemistry can be adopted to react the free amine side chains of the hydrogel with the carboxylic end of the DHBA. Since the concentration of DHBA hydroxyl groups may be critical for enhanced binding of iron, the choice of appropriate PAAm-DHBA ratio was important for obtaining hydrogels with high iron affinity.

### Conditional Stability Constant of Fe(III)-Hydrogel.

The ligand competition method is widely used for the determination of stability constants of both soluble iron(III)-ligand complexes and cross-linked polymeric chelators. A decrease in the concentration of DHBA results in a decrease of the conditional stability constant. As the concentration of functional groups incorporated in hydrogels decreases, the binding capacity of the hydrogel decreases as well. Chelating properties of a polymeric chelator have also been shown to be affected by steric hindrance between the ligand and the polymeric matrix, but in the case of the cross-linked PAAm-DHBA hydrogels there may be little inference by the polymer backbone in the iron chelation process.

### Selectivity of the PAAm-DHBA Hydrogels.

Since PAAm-DHBA hydrogels possessed a high affinity for Fe(III), it was anticipated that cross-linked PAAm-DHBA hydrogels may also possess an improved selectivity for Fe(III) over other metal ions. Copper(II), zinc(II), and manganese(II) are all present in biological tissues and in food. As these three metals are essential for life, it is important that the hydrogels designed in this study possess much lower affinities for this group of divalent cations.

Synthesis at various crosslinker:polymer ratios facilitated identification of an acceptable range of swelling indices for biomedical applications while maintaining an acceptable reaction yield. These values are known to provide sufficient mechanical integrity and chemical stability after oral administration, based upon research reports and on data for the FDA-approved product, Renagel®.

### Swelling Studies.

The swelling behavior of hydrogels can be studied using buffered solutions (sodium hydroxide as a buffering agent) with fixed ionic strength (0.5 M). A historic protocol was used for making buffer solution with a known ionic strength. Dried samples with known weights were placed in a solution of defined pH at room temperature. Samples were taken from the solution after reaching equilibrium. The swelling indexes (SI) were calculated using the following equation:

$$SI = \frac{{w}_{s} - {w}_{d}}{{w}_{d}} \qquad ( 1 )$$

where Wₛ is the weight of the swollen hydrogel at an equilibrium state, and W_{d} is the weight of the dried hydrogel.

### Binding Kinetics.

Determination of the kinetics of metal absorption is useful for elucidating the reactivity of the cross-linked polymeric chelators and evaluating their potential for chemical and biomedical applications. The kinetics of metal binding can be monitored using a known initial concentration of metal solution (2 mg/mL, FeCl₃) in the presence of a known mass of dry hydrogels.

Known concentrations of ferric chloride and ferrous chloride solutions (0.25, 0.5, 1, 2, 2.5) mg/mL can be prepared. Binding experiments may be carried out by taking 20 mL of metal solutions in 125 mL volumetric flasks, and solutions adjusted to the desired pH while maintaining iron concentration. Next, a known mass of cross-linked hydrogel is added to the mixture and is held at room temperature for 2 hours or until equilibrium is reached. The solutions are then filtered and the filtrates are analyzed for metal concentration.

Rapid absorption behavior is important in biomedical application especially for treatment of acute metal poisoning. To derive the rate constant and binding capacity, the kinetic data can be modeled with pseudo-first-order (Lagergren model) and pseudo-second-order (Ho model) kinetic models which are expressed in their linear forms as:

$$log ( {q}_{e} - {q}_{t} ) = log ( {q}_{e} ) - \frac{{k}_{1}}{2.303} t \\ \frac{t}{{q}_{t}} = \frac{1}{{q}_{e}^{2} {k}_{2}} + \frac{t}{{q}_{e}}$$

where k₁ (L/min) and k₂ (g/mg min) are pseudo-first-order and pseudo-second-order rate constants, respectively.

$$\overline{n} = \frac{{C}_{H α} - ( {H}^{+} ) + ( {OH}^{-} )}{{C}_{H}}$$

Here, C_{H} is the concentration of hydrogel.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited herein are incorporated herein by specific reference.

### Alternative Synthesis, NHS-Activated DHBA.

A solution of DHBA (0.5 kg, 3.27 mol), NHS (0.75 kg, 6.54 mol) in 1.7 L of DMF was mixed with a suspension of EDC (0.56 kg, 2.9 mmol) in 1.7 L of DMF. The mixture was stirred until completion of the reaction and used for the next reaction step.

### Preparation of DHBA Modification of Hydrogel.

PAAm with an average molecular weight of 15 kDa-18 kDa (2.5 kg of a 50% solution in water, 13.1 mol) is further diluted with 1.98 kg of water before the NHS-activated DHBA solution is added, followed by an additional 0.125 kg of water, followed by a solution of BMA (0.002 kg, 0.01 mol) in DMF/water (0.25 L each) and triethylamine (2.35 kg, 23.2 mol). The reaction mixture is stirred for 24 hours. The resultant cross-linked polymers were then washed with 0.1 M sodium hydroxide, acetonitrile/water, iso-propanol and isopropanol/water. The resulting polymer was than milled and sieved to archive the target particle size distribution.

What is claimed is:

1-48. (canceled)

49. A composition comprising:
(i) a polymeric chelator comprising a plurality of polyamine polymer backbone chains and one or more chelators; and
(ii) an agent selected from an antacid, a histamine H2-receptor antagonist, a proton pump inhibitor, and a combination thereof,
wherein the one or more chelators are covalently coupled to one or more primary and/or secondary amines of at least one of the plurality of polyamine polymer backbone chains; and
wherein the plurality of polyamine polymer backbone chains are cross-linked with a plurality of cross-linkers.

50. The composition of claim 49, wherein each of the plurality of polyamine polymer comprises a polyamine polymer having a weight average molecular weight of 1-50 kDa.

51. The composition of claim 49, wherein the each of the polyamine polymer backbone chains comprises monomeric units each having the structure: 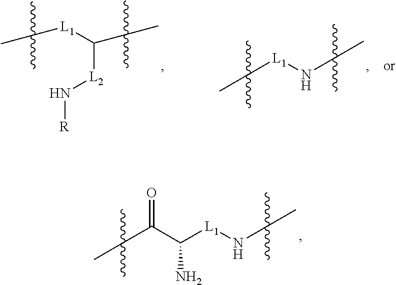
wherein
L₁ is C₁-C₆alkylene;
L₂ is a bond or C₁-C₆alkylene; and
R is H or C(O)R″, in which R″ is H, C₁-C₆ alkyl, or C₆-C₁₂ aryl.

52. The composition of claim 49, wherein the plurality of polyamine polymer backbone chains each comprise polyallylamine.

53. The composition of claim 49, wherein the plurality of polyamine polymer backbone chains each comprise poly(L-lysine).

54. The composition of claim 49, wherein the agent is an antacid.

55. The composition of claim 54, wherein the antacid is CaCO₃, NaHCO₃, MgCO₃, Mg(OH)₂, MgO, Al(OH)₃, or simethicone.

56. The composition of claim 49, wherein each of the plurality of cross-linkers is independently of structure: 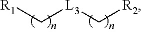
wherein
R₁ and R₂ are independently selected from: 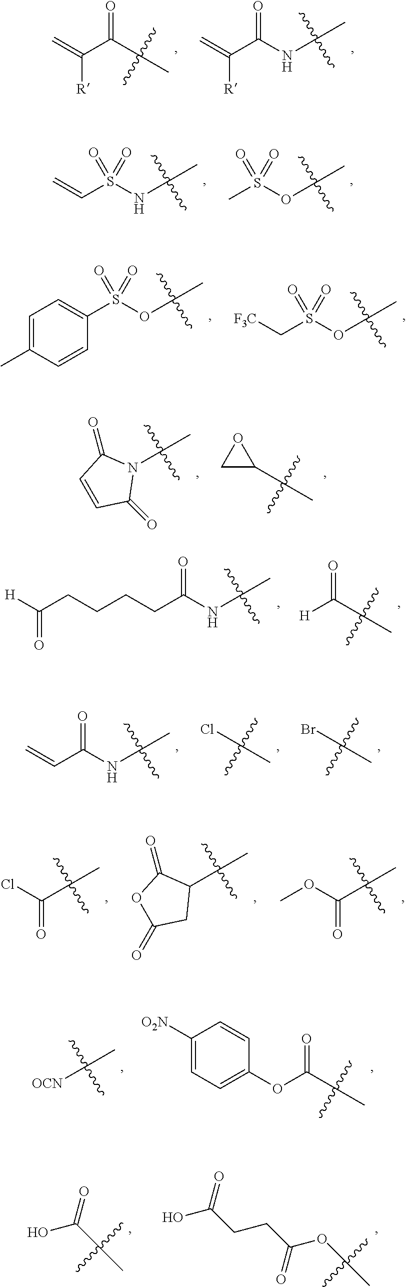 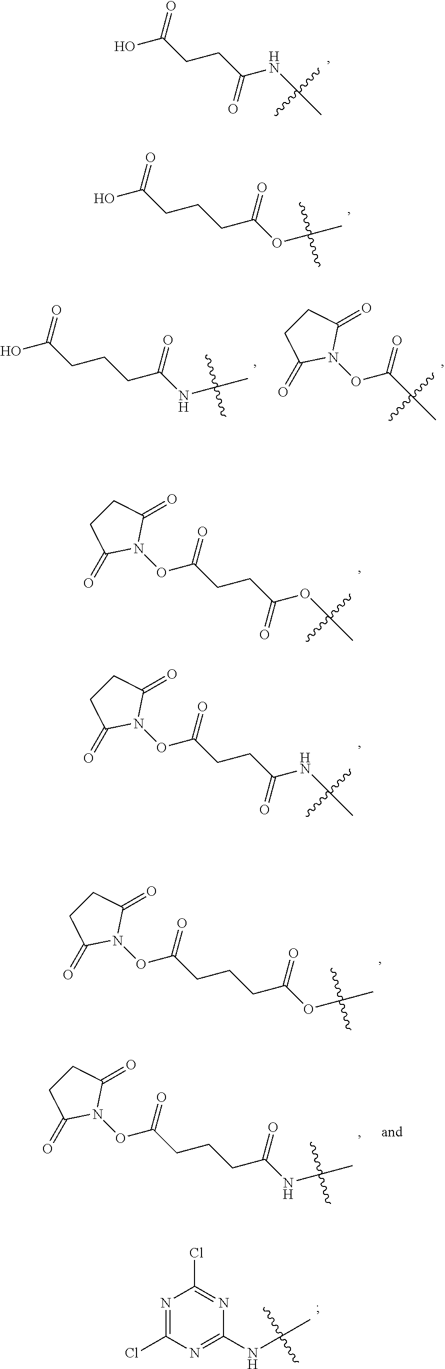
R′ is H or C₁-C₆alkyl;
n is 0, 1, or 2; and
L₃ is a bond, C₁-C₆alkylene, C₁-C₆ heteroalkylene, C₃-C₈ cycloalkylene, C₆-C₁₄ arylene, or 5- or 6-membered heterocyclylene or polyethylene glycol.

57. The composition of claim 56, wherein each of the plurality of cross-linkers is of structure:

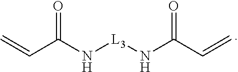

58. The composition of claim 57, wherein the polymeric chelator comprises a plurality of groups each having the structure:

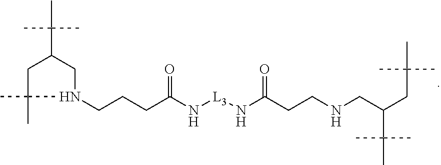

59. The composition of claim 57, wherein L₃ is methylene.

60. The composition of claim 56, wherein each of the plurality of cross-linkers is of structure:

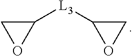

61. The composition of claim 60, wherein the polymeric chelator comprises a plurality of groups each having the structure:

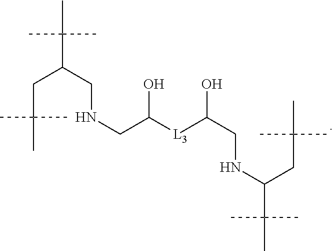

62. The composition of claim 56, wherein L₃ is polyethylene glycol.

63. The composition of claim 62, wherein the polyethylene glycol has a number average molecular weight of 200 Daltons to 6000 Daltons.

64. The composition of claim 63, wherein the polyethylene glycol has a number average molecular weight of 400 Daltons to 2500 Daltons.

65. The composition of claim 64, wherein the polyethylene glycol has a number average molecular weight of 800 Daltons to 2200 Daltons.

66. The composition of claim 49, wherein each of the plurality of cross-linkers is selected from:

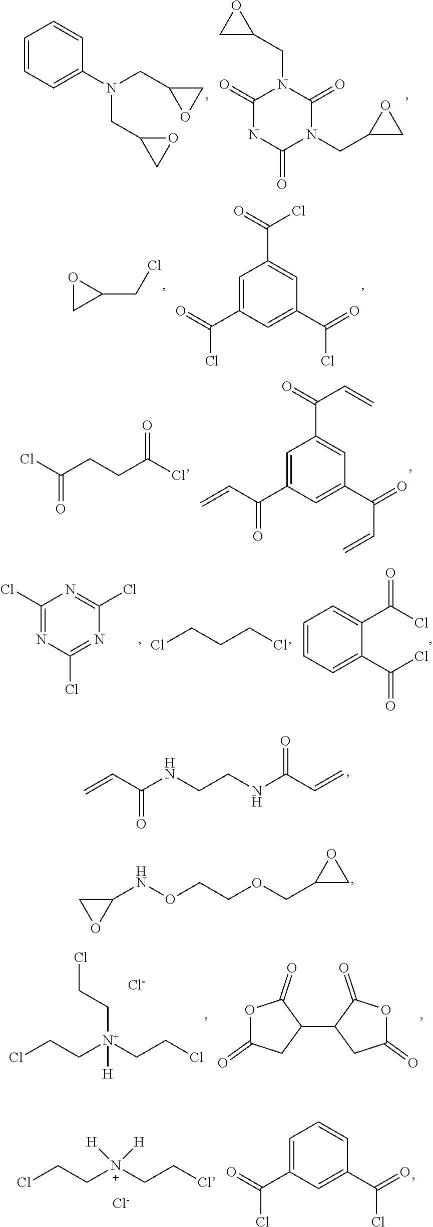

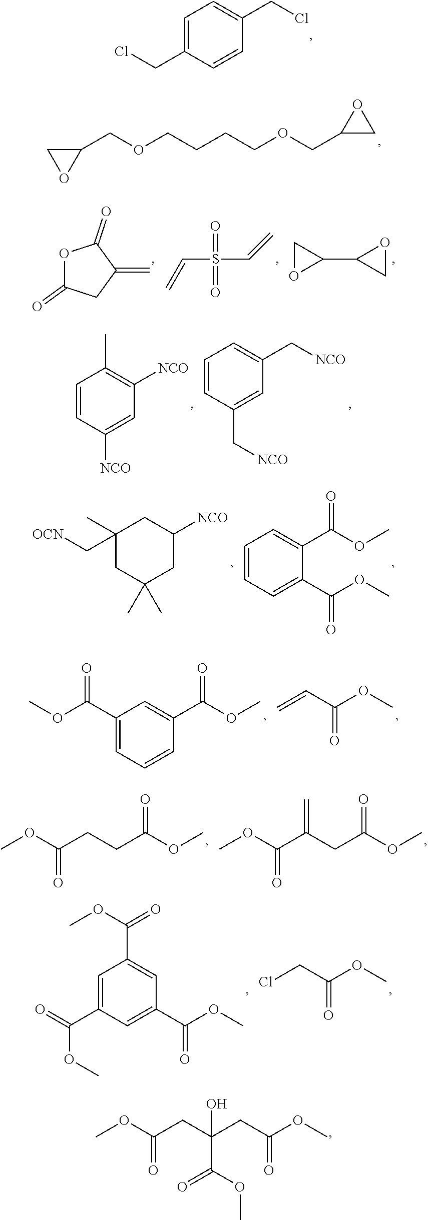

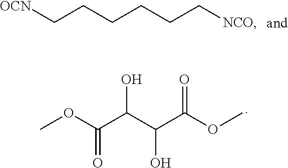

67. The composition claim 49, wherein each of the plurality of cross-linkers is a hydrophilic cross-linker.

68. The composition of claim 49, wherein the plurality of cross-linkers comprise individual cross-linkers with a number average molecular weight of 200 Daltons to 6000 Daltons.
